(19) United States
(12) Patent Application Publication
(10) Pub. No.: US 2005/0059137 A1
(43) Pub. Date: Mar. 17, 2005

(54) Bio-support and preparing method of the same

(76) Inventor: Younghoon Lee, Daejeon-city (KR)

Correspondence Address: FISH & RICHARDSON PC, 225 FRANKLIN ST, BOSTON, MA, 02110, US

(21) Appl. No.: 10/399,168

(22) PCT Filed: May 31, 2001
(86) PCT No.: PCT/KR01/00916

(30) Foreign Application Priority Data
Oct. 14, 2000 (KR) 2000/60540

(51) Int. Cl. C12M 1/34; A61K 9/14

(52) U.S. Cl. 435/287.2; 528/230; 424/489; 427/2.11

(57) ABSTRACT

The present invention relates to a bio-support and preparing method of the same, and more particularly, to a method for immobilizing the bio-polymer on a slide glass when the bio-chip is prepared. The preparing method of bio-support comprises the following steps, (a) forming a dendrimer monolayer by generating Schiff base between aldehyde groups on a silylated slide and dendrimer, and (b) converting non-reacted aldehyde groups to alcohol groups on the slide (a). The bio-supports of the present invention provide three-dimensional space for effective immobilization of bio-polymers. Also, the bio-supports can promote complementary interactions between bio-polymers.

### BACKGROUND OF THE INVENTION

(a) Field of the Invention

The present invention relates to a bio-support and preparing method of the same, and more particularly, to a method for immobilizing the bio-polymer on a slide glass when the bio-chip is prepared.

(b) Description of the Related Art

The recent microarray system based in hybridization is a widely used technique and has numerous applications. The microarray system employed in various fields has gradually developed from the basic concept, that is, labeled nucleic acid molecules could be used to detect nucleic acid molecules fixed on solid surfaces.

The main research on the DNA chip has recently been carried out in USA and part of the research has been carried out in Europe. Further, venture companies involved in fabrication and application of the DNA chip have emerged and large enterprises including Molecular Dynamics, Motorola and so on support the new industry. Up to 1998, an array that researchers could purchase was a form of immobilizing genes on the filter. NEN life Science provides an array of immobilizing a number of 2,400 human cDNA oligonucleotides on a slide glass. Affymetrix and Incyte offer DNA chips for human EST, mouse, yeast and bacteria, and Clontech offers a cDNA array of slide type. All these goods are fabricated by immobilizing oligonucleotides on a two-dimensional surface. That is, after the surface of glass is treated with poly-lysine, DNA is immobilized to poly-lysine on glass by a crosslinking reaction. Alternatively, after SAM (self assembled monolayer) of an aldehyde or an amine group is prepared on glass, DNA is bound to that glass. Such methods can immobilize nucleic acids from short oligonucleotide to long length cDNA, but the application is limited by the surface density of the attached nucleic acids and hybridization efficiency between target and probe nucleic acids.

Many studies were carried out to develop various solid support, such as polyacrylamide gel pad, gelatin pad or agar film on glass, which could eliminate the limiting factors of the surface density and hybridization efficiency. Especially polyacrylamide gel provides a three dimensional solid support with a great capacity of immobilization (Rehman, F. N., Audeh, M., Abrams, E. S., Hammond, P. W., Kenney, M., and Boles, T. C. (1999) Nucleic Acids Res., 27, 649-655; Guschin, D., Yershov, G., Zaslavsky, A., Gemmell, A., Shick, V., Proudnikov, D., Arenkov, P., and Mirzabekov, A. (1997) Anal. Biochem., 250, 203-211), but has a low hybridization yield due to lack of space between solid support and oligonucleotides. In order to improve the hybridization yield, various linkers connecting between immobilized nucleic acids and solid supports were introduced (Guo, Z., Guilfoyle, R. A., Thiel, A. J., Wang, R., and Smith, L. M. (1994) Nucleic Acids Res., 22 5456-5465; Shchepinov, M. S., Case-Green, S. C., and Southern, E. M. (1997) Nucleic Acids Res., 25, 1155-1161). However, most of methods including modifying materials are very complex and can be applied only under specific conditions.

### SUMMARY OF THE INVENTION

It is an object of the present invention to provide a bio-support that can immobilize bio-polymers such as nucleic acid, protein and antibody.

Also, it is an object of the present invention to provide a preparing method of bio-supports that can immobilize bio-polymers such as nucleic acid, protein and antibody.

In order to achieve these objects, the present invention provides a bio-support comprising (a) slide glass including aldehyde groups on surface; and (b) dendrimer binding to the aldehyde group of (a).

Also, the present invention provides a method of preparing a bio-support comprising the following steps: (a) forming dendrimer monolayer by generating Schiff base between aldehyde groups on silylated slide and amine groups of dendrimer; and (b) converting non-reacted aldehyde groups to alcohol groups on slide (a) by NaBH₄.

Also, the present invention provides a bio-chip with bio-polymers selected from the group consisting of nucleic acid, protein, peptide, antibody, and chemicals, immobilized to the bio-support of the above.

### BRIEF DESCRIPTION OF THE DRAWINGS

FIG. 1 shows a dendrimer generation 3 employed in a bio-support of the present invention.

FIG. 2 shows a dendrimer generation 4 employed in a bio-support of the present invention.

FIG. 3 is an illustration showing the preparation process of dendrimeric solid support.

FIG. 4 shows a dendrimeric bio-support of the present invention.

FIG. 5 shows a pathway of preparing DNA chip.

FIG. 6 shows a pathway of preparing protein chip.

FIG. 7 is a densitometric picture obtained after immobilization of oligonucleotides on a dendrimeric bio-support and autoradiography.

FIG. 8 is a picture showing the hybridization yield of the dendrimeric bio-support and autoradiography.

### DETAILED DESCRIPTION OF THE PREFERRED EMBODIMENTS

The present invention will now be explained in more detail.

In the development of the microarray system for clinical diagnostics, inventors studied an immobilizing method of polymer (nucleic acid, protein, and so on) on the surface of glass that was at the core of the bio-chip preparing method, and developed a bio-support and the bio-chip.

The bio-support of the present invention contains dendrimer to immobilize bio-polymers onto the surface of the slide glass with a three dimensional structure. Thus, after binding dendrimer to aldehyde groups of the slide, bio-polymers are immobilized to the dendrimer. The dendrimer has been studied from the middle of the 1980s and an investigative focus on synthetic method, physical and chemical properties has been made. Most studies for the dendrimer have been carried out for on plasticizer, liquid crystal, layers and drug-delivery; however, the dendrimer is still not commonly used

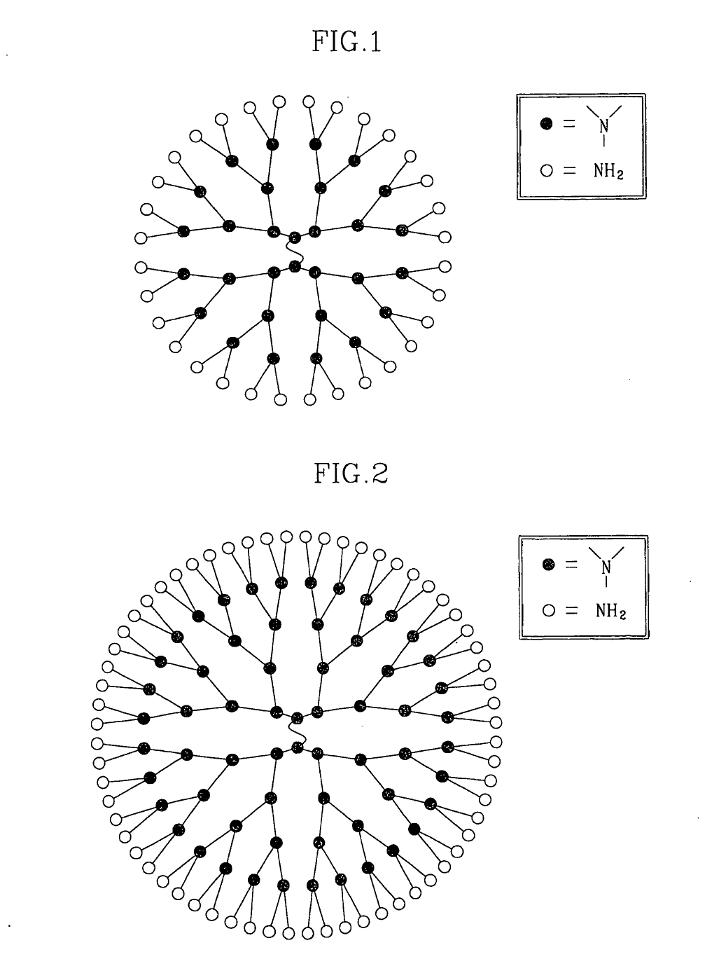

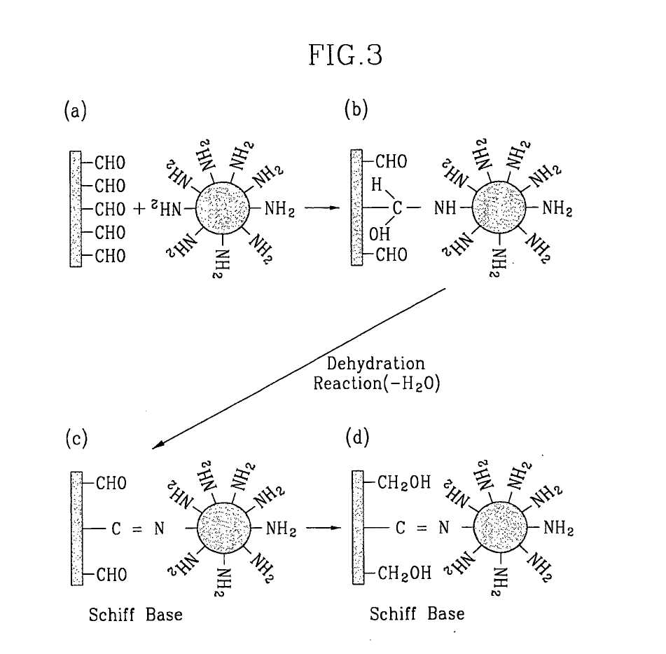

The polyamidoamine (PAMAM) dendrimer of the present invention as shown in FIG. 1 contains a radial shape that branches from a nucleophilic core, or an electrophilic core, to an amidoamine, and a three-dimensional sphere-like structure. The PAMAM dendrimer generation 3 in FIG. 2 has 40 Å diameters and 32 amine groups.

The amine group of PAMAM dendrimer increases twice and diameter increases 10 Å diameter per generation. FIG. 2 also shows a PAMAM dendrimer generation 4 including 64 amino groups. The dendrimer of the present invention provides a unique structure and a three-dimensional structure with branched amine groups. Also, the dendrimer of the present invention is supposed to be of an ellipsoidal shape (Tokuhisa, H., Zhao, M., Baker, L. A., Phan, V. T., Dermody, D. L., Garcia, M. E., Peez, R. F., Crooks, R. M., and Mayer, T. M. (1998) J. Am. Chem. Soc. 120, 4492-4501; Bliznyuk, V. N., Rinderspacher, F., and Tsukruk, V. V. (1998) Polymer, 39, 5249-5252).

Preferable dendrimers of the present invention are dendrimer generation 1 to dendrimer generation 8, more preferably, dendrimer generation 2 to dendrimer generation 6, and most preferably dendrimer generation 3 to dendrimer generation 4.

Bio-polymers immobilized to the dendrimer can be selected from the group consisting of nucleic acids, protein, peptide, chemicals, and antibody; nucleic acid and protein are preferable, and nucleic acid is most preferable.

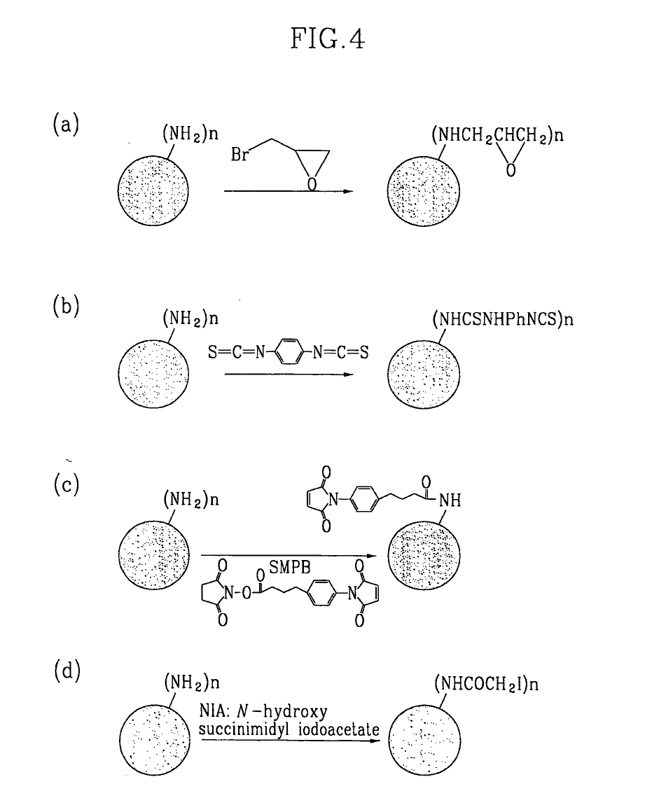

A model of bio-support of the present invention is represented as FIG. 3(d), and contains dendrimer bound to an aldehyde group fixed on the surface of a slide glass.

Also, a bio-support of the present invention contains a linker connected with an amine group of dendrimer. The linker is a connecter, which can immobilize bio-polymers on a solid support easily and the linker can be selected from groups consisting of chemicals represented by the following formula 1, formula 2 (1,4-phenylene diisothiocyanate; PDC), formula 3 and n-hydroxysuccinimidyl iodoacetate (NIA)

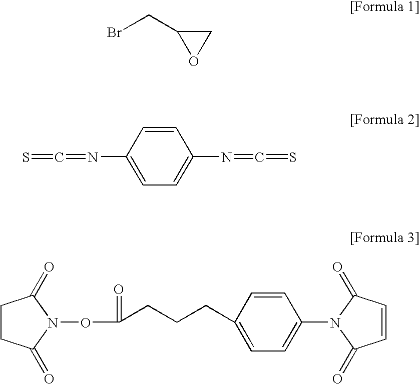

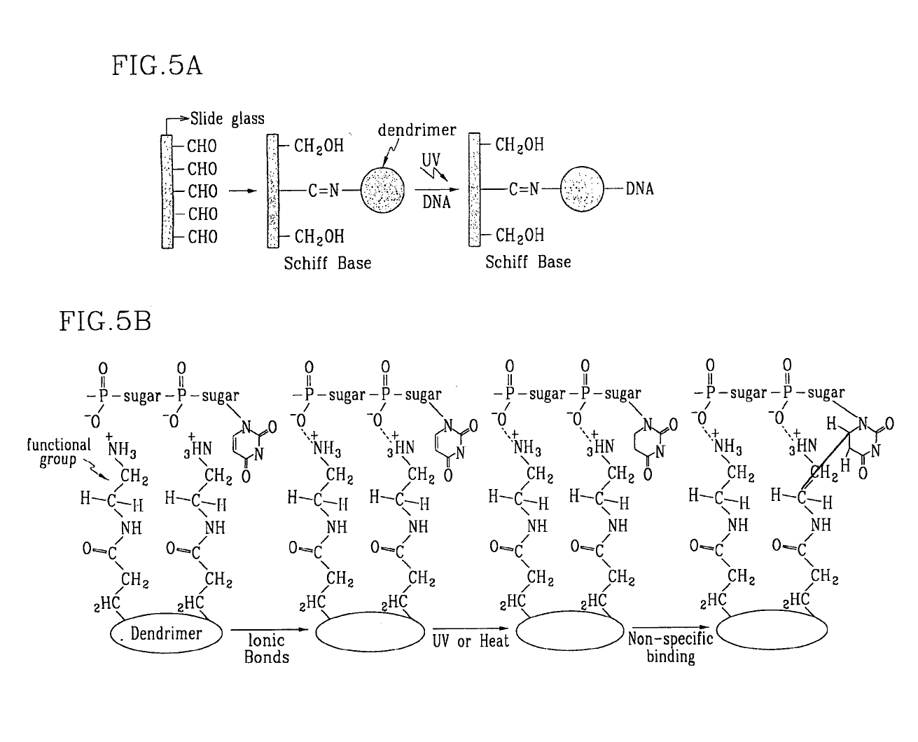

The bio-supports including linkers are shown in FIG. 4. FIG. 4(a) is the bio-support containing linker of formula 1, FIG. 4(b) is the bio-support containing PDC of formula 2, FIG. 4(c) is the bio-support containing linker of formula 3, and FIG. 4(d) is the bio-support containing NIA linker. The bio-supports shown in FIG. 4 can immobilize nucleic acid, protein, peptide, antibody and so on.

The diameter of dendrimer increase about 17 Å by the coupling of PDC. According to this fact and high dendrimer coverage, the surface density of active thiocyanate groups is about 0.06 nmol/cm² and the average distance between neighboring thiocyanates is about 18 Å. The 18 Å is nearly the same as 18 to 20 Å of the diameter of DNA helix. This distance by this invention contrasts with 5 to 10 Å of distance between terminal functional groups of the solid support with two dimensional structures which showed 0.3 nmol/cm² of surface density. (Guo, Z., Guilfoyle, R. A., Thiel, A. J., Wang, R., and Smith, L. M. (1994) Nucleic Acids Res., 22, 5456-5465; Matson, R. S., Rampal, J. B., and Coassin, P. J. (1994) Anal. Biochem., 217, 306-310). Therefore, dendrimer of bio-supports of the present invention is suitable for immobilizing nucleic acids.

Although the bio-support of the present invention contains-functional terminal groups (i.e., the number of thiocyanate groups in case of the bio-support containing PDC-dendrimer) fewer than the other support with two-dimensional structures, the bio-support of the present invention can immobilize oligonucleotide with a high efficiency due to the three-dimensional position of thiocyanate and the ideal distance between functional groups.

Also, the present invention provides a preparing method of the bio-support. The preparing method of the bio-support is shown in FIG. 3 and is explained in more detail.

A slide with aldehyde groups on surface was used as a bio-support material of the present invention. The slide prefers silylated slide. The commercial silylated slide has reactive aldehyde groups on surface. Firstly, the aldehyde groups of silylated slide were reacted with dendrimer and then schiff base between the aldehyde groups and the dendrimer was generated. Thus, a slide including the dendrimer monolayer on surface was generated. Next, the slide was performed with hydrogenation reaction by NaBH₄, to convert non-reacted aldehyde groups to alcohol groups. Bio-support was prepared by the above method.

Also, the preparing method of bio-support further contains a connecting step of linker after the converting step.

In the forming step of dendrimer monolayer, slide glass is reacted with methanol containing 0.5% of dendrimer and thus, aldehyde on slide glass is reacted with dendrimer. After the reaction of dendrimer with aldehyde groups as shown in FIG. 3(b), Schiff base is generated by the dehydration reaction of FIG. 3(c).

In the converting step, non-reacted aldehyde groups were transformed to alcohol and the bio-support of FIG. 3(d) was formed.

The connecting step of linker generates a binding between the amine group of dendrimer and linker. The linker is preferably selected from the group consisting of chemicals represented by formula 1, formula 2, formula 3, and n-hydroxysuccinimidyl iodoacetate (NIA). The connecting method of linker prefers a known method. (Chrisey, L. A., Lee, G. U. and O. Ferrall, C. E. Nucleic Acids Res. (1996) 24, 3031-3039, Singh, P. Bioconjugate Chem. (1998) 9, 54-63 Singh, P. Bioconjugate Chem. (1998) 9, 54-63) FIG. 4 shows the bio-supports including linkers.

Also, the present invention provides a bio-chip using the above bio-supports. The bio-chip is preferable DNA chip, protein microarray, antibody support, biosensor, and combinatorial array.

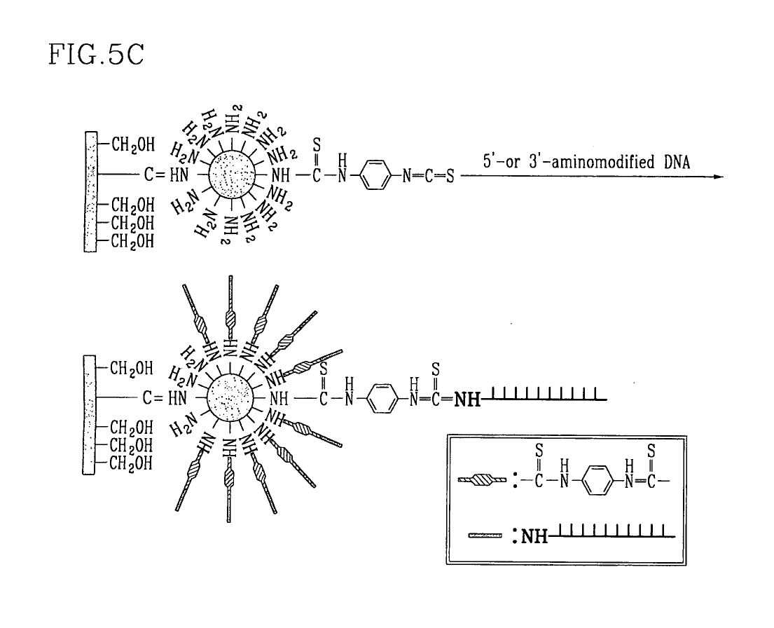

The bio-chip contains the bio-support of the present invention and bio-polymers immobilized to the bio-support. More particularly, a bio-polymer is immobilized to amine group of dendrimer bound to aldehyde on slide glass. The fabrication method of bio-chip is preferable to perform general UV-crosslinking or heating reaction. FIG. 5 shows a preparation process of the DNA chip.

The present invention further contains a bio-chip using bio-support including linker. The bio-chip comprises the following steps: (a) reacting dendrimer with aldehyde groups on slide glass, (b) converting non-reacted aldehyde groups to alcohol group on slide, (c) binding a linker to the amine group of dendrimer made in (b), and (d) immobilizing bio-polymers to the linker made in (c). DNA chip can be prepared by a UV-crosslinking reaction represented in FIG. 5(a) and (b) or a reaction represented in FIG. 5(c).

FIG. 5(c) shows a preparation process of DNA chip by using a linker and oligonucleotides that are modified oligonucleotides with amine or thiol at the 5′ or 3′ terminus. The size of the oligonucleotides prefers that of generally used oligonucleotides in DNA chip. In case of the use of the thiol-modified oligonucleotides, DNA chip can be prepared by immobilizing them to the bio-support with other linkers than PDC.

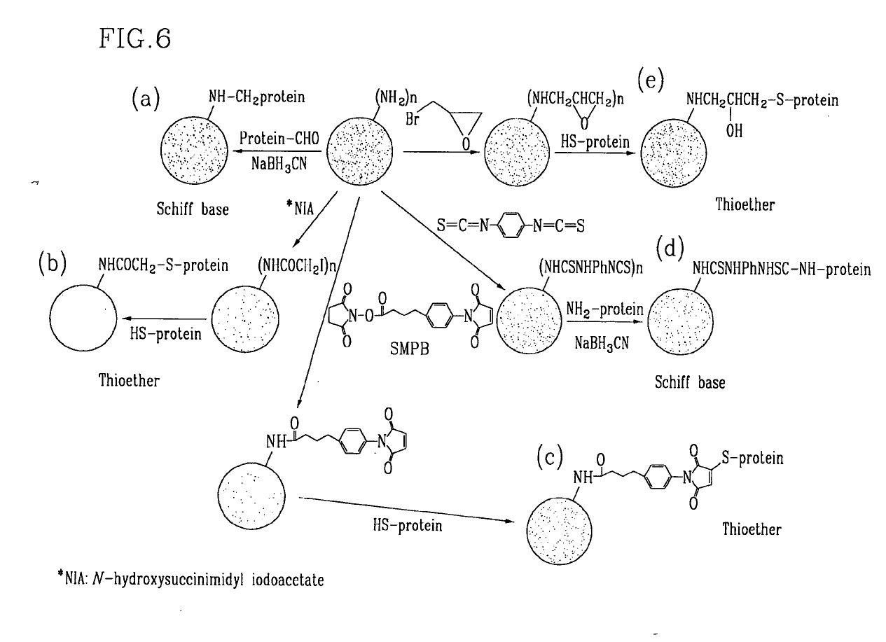

Also, in protein chip of the present invention, FIG. 6 shows an example for the preparation process of protein chip. The protein chip can be prepared by immobilizing protein to the dendrimeric bio-support (FIG. 6(a)) directly or with linkers (FIG. 6(b, c, d)).

The present invention will be explained in more detail with reference to the following Examples. However, the following Examples are to illustrate the present invention and the present invention is not limited to them.

### EXAMPLE 1

All chemicals are purchased from Sigma-Aldrich (USA) unless stated otherwise, and silylated slide glass is purchased from Cel Associates (USA). Oligonucleotides are synthesized at Genotech (Taejon, Korea) and dendrimers of generation 3 and generation 4 are purchased from Sigma-Aldrich.

Manufacturing of Bio-Support

Silylated slides were washed and immersed in methanol containing 0.5% of PAMAM dendrimer (generation 3, FIG. 1) for 1-2 days. Thus, the surface of slide glass was formed with the monolayer of dendrimer by generating Schiff base between amine groups of dendrimer and aldehyde groups of SAM (self assembled monolayer) on the slide surface. The remaining non-reacted aldehyde groups on slide glass were converted to alcohol groups by adding sodium borohydride. After the reaction, the slide glass was washed three times and dried for 30 mins under vacuum.

### EXAMPLE 2

Oligonucleotides were dissolved in 3×SSC(SSC: 150 mM NaCl, 15 mM sodium acetate, pH 7.0) and spotted on a bio-support constructed by the same method as described in Example 1. The spotted solution was dried and cross-linked with UV-crosslinker (60 mJ)

### EXAMPLE 3

The experiment was performed by the same method as described in Example 1, except that dendrimer of generation 4 was used instead of generation 3.

### EXAMPLE 4

After bio-support was prepared by the same method as described in Example 1, bio-support containing a linker was further manufactured. Firstly, in order to conjugate the linker to dendrimer, the dried slide glass was treated with 0.2% of 1,4-phenylene diisothiocyanate (PDC, Aldrich) in 10% of pyridine/dimethyl formamide for 3 hours under argon gas. After the reaction, the slide glass was washed with methanol and stored in a desiccator until use.

### EXAMPLE 5

After bio-support was prepared by the same method as described in Example 3, bio-support containing the linker was further manufactured as described in Example 4.

### COMPARATIVE EXAMPLE

The silylated slide that has aldehyde SAM on surface of slide glass was used as a support for DNA chip.

### Experiment

Immobilization of Oligonucleotide

Bio-supports prepared by Example 4, Example 5 and Comparative example were used. The oligonucleotide used was 5′ CCGACCGGAATAAAT-NH₂-3′, which had an amine group at the 3′-terminus. To monitor the immobilization efficiency of the oligonucleotide, the oligonucleotide was labeled with ³²P at the 5′-terminus. The oligonucleotide of 10 pmol was labeled with of [γ-³²P]ATP(>6,000 Ci/mol, 10 mCi/ml) and T4 polynucleotide kinase at 37° C. for 30 min. The reaction was stopped by heating at 95° C. for 2 min and then the labeled oligonucleotide was purified by a G-50 spin column. The concentration of the oligonucleotide was adjusted to 0.005 pmol/μl, 0.001 pmol/μl, and 0.03 pmol/μl. The 0.5 μl solution of each concentration was spotted on the bio-support prepared by Example 4, Example 5, or Comparative example and dried for 16 hours at room temperature. The dried bio-support was washed with water, 3 N NH₄OH and 1×SSPE (150 mM NaCl, 10 mM NaH₂PO₄, pH 7.4, 1 mM EDTA) containing 0.2% of SDS to remove the unbound oligonucleotides. The surface density of immobilizing oligonucleotide on the bio-support was determined by scanning the slide with BAS1500 (FUJI, JAPAN).

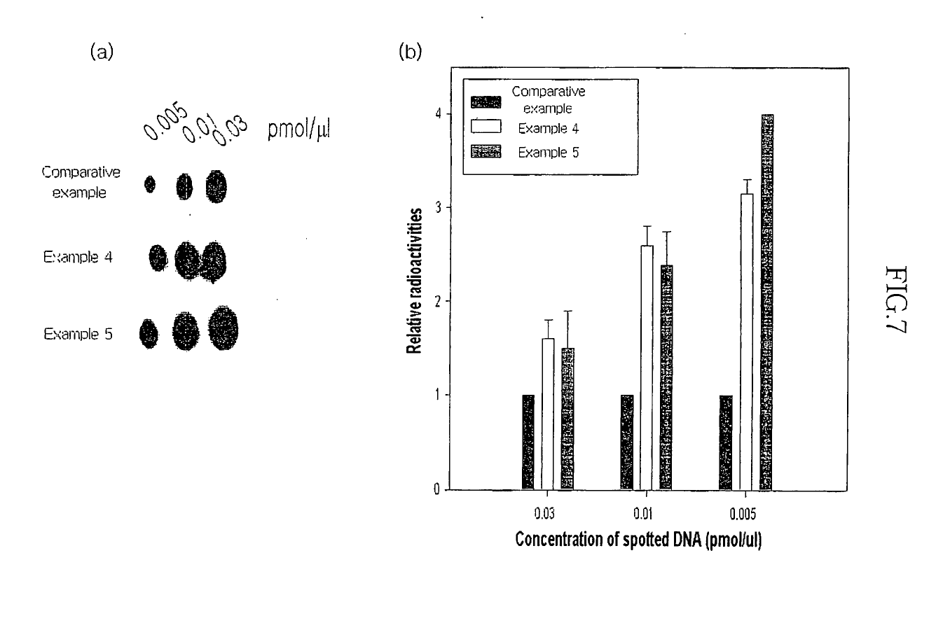

FIG. 7 is an autoradiograph (a) obtained after immobilization of the oligonucleotide on the dendrimeric bio-support and its bar graph (b). It shows that the radioactivity is proportional to the concentration of the oligonucleotide. The surface density of the bio-support prepared by Example 4 or Example 5 was 2-3 times higher than that of Comparative example. This result shows that oligonucleotide immobilizes well on the bio-support compared with the support containing only high-density aldehyde SAM. Also, when the bio-support of Example 5 was compared with Example 4, the immobilizing efficiencies of Example 4 and Example 5 are almost same although the bio-support of Example 5 was expected to carry two times more oligonucleotides than the bio-support of Example 4.

Analysis of Hybridization Efficiency

An oligonucleotide was immobilized on each of the bio-supports prepared by Example 4, Example 5 and Comparative example, and the hybridization efficiency was analyzed with the complementary oligonucleotide.

The unlabeled target oligonucleotide 5′-CCGACCGGMTAAAT-NH₂-3′ was immobilized on the bio-supports and the complementary oligonucleotide 5′-ATTTATTCCGGTCGG-3′ labeled with [γ-³²P]ATP at the 5′-terminus was used as a probe. The slide glass immobilized with the target oligonucleotide was pre-hybridized for 2 hours in 5×SSPE containing 0.2% of SDS and hybridized with the probe oligonucleotide to a final concentration of 2 pmol/ml at 42° C. for 16 hours. After the hybridization reaction, the unhybridized probe was removed by washing with 1×SSPE containing 0.2% of SDS followed by 0.1×SSPE containing 0.2% of SDS for 30 mins at 38-40° C. The hybridization efficiency was measured by scanning the slide with BAS1500.

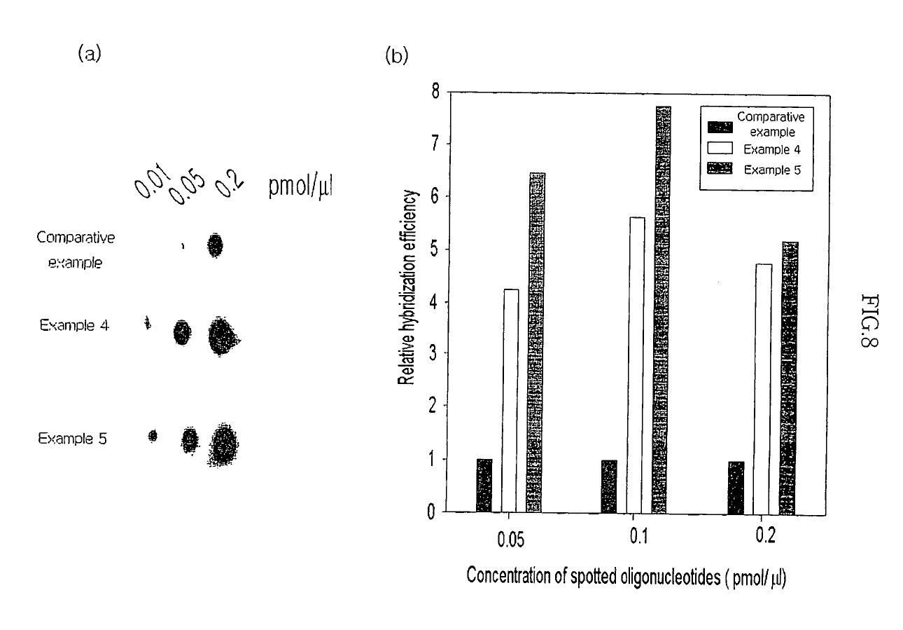

FIG. 8 is an autoradiograph (a) that shows the hybridization efficiency of DNA-chip prepared with the bio-support and its bar graph (b). The bio-supports of Example 4 and Example 5 showed the hybridization efficiency the maximum eight times more than the support of Comparative example. Considering that the bio-supports of Example 4 and Example 5 can immobilize oligonucleotides only two to three times more than that of Comparative example, this result shows that the bio-support of Example 4 and Example 5 can provide the high hybridization yield in addition to the improved oligonucleotide immobilization. The high efficiency of hybridization can be explained by the fact the bio-support of the present invention provides three-dimensional spacing enough for the incoming probe nucleotide to form a hybrid with the immobilized oligonucleotide. The flexibility of the PDC linker between dendrimer and the oligonucleotide can also contribute to the hybridization yield.

In many cases of DNA microarray using modified glass slides to improve the surface density of immobilized nucleic acids, there is also the increase of non-specific binding background signals on the activated surface of slides. The high level of background signals decreases the sensitivity for analysis of microarray.

It is possible that the bio-support of the present invention can leave positively charged amine groups, which could interact electrostatically with negatively charged nucleic acids. However, in FIG. 7 and FIG. 8, the bio-supports of Example 4 and Example 5 did not show any non-specific binding on the surface. This indicates that all amine groups of dendrimer of the bio-supports were converted to thiocyanate groups by reacting with PDC. As a result of the conversion of all amine groups to those competent for immobilization, the bio-supports of the present invention were able to both to immobilize the oligonucleotide with high efficiency and cause the decrease of non-specific binding.

As it is defined in detail above, the bio-supports of the present invention contain dendrimer conjugated to aldehyde groups on glass slides, and generate 3-demensional space to immobilize bio-polymers with high efficiency. Also, the bio-supports can be generally used for preparing bio-chips. When DNA chips were prepared using the bio-supports, the DNA chips can get high complementary binding.

1. A bio-support comprising;
(a) Slide glass including aldehyde groups on surface; and
(b) Dendrimer binding to the aldehyde group of (a).

2. The bio-support according to claim 1, wherein the bio-support further contains linker binding to amine group of the dendrimer.

3. The bio-support according to claim 1, wherein the linker is selected from groups consisting of chemicals represented by the following formula 1, formula 2, formula 3 and n-hydroxysuccinimidyl iodoacetate (NIA).

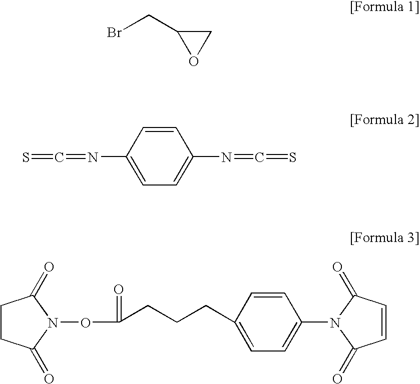

4. The bio-support according to claim 1, wherein the dendrimer is dendrimer generation 1 to dendrimer generation 8.

5. A method of preparing bio-support comprising the following steps:
(a) Forming dendrimer monolayer by generating Schiff bases between aldehyde groups on the silylated slide and amine groups of dendrimer; and
(b) Converting non-reacted aldehyde groups on the slide a) to alcohol by NaBH₄.

6. The method of preparing bio-support according to claim 5, wherein the method further comprises a step of binding a linker onto amine groups of dendrimer after the converting step.

7. The method of preparing bio-support according to claim 6, wherein the linker is selected from the group consisting of chemicals represented by formula 1, formula 2, formula 3 and n-hydroxysuccinimidyl iodoacetate (NIA).

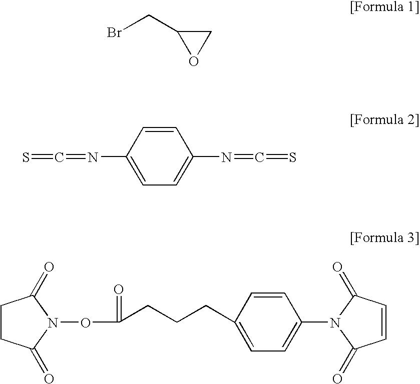

8. Bio-chips with bio-polymers, which are selected from the group consisting of nucleic acid, protein, peptide, antibody and chemicals, immobilized to the bio-support of claim 1.


SEQUENCE LISTING (2 sequences)

SEQ ID NO: 1 - DNA, 15 bp, Artificial Sequence
  misc_feature: this sequence has an amidated  3′-terminus
ccgaccggaataaat

SEQ ID NO: 2 - DNA, 15 bp, Artificial Sequence
  misc_feature: this sequence is labeled with [gama-32P]ATP at 5′-terminus
atttattccggtcgg